(19) United States
(12) Patent Application Publication
(10) Pub. No.: US 2017/0138956 A1
(43) Pub. Date: May 18, 2017

(54) METHODS OF DETERMINING SEROTYPE SPECIFICITY OF AN ANTIBODY

(71) Applicant: THE REGENTS OF THE UNIVERSITY OF CALIFORNIA, Oakland, CA (US)

(72) Inventors: Eva HARRIS, Berkeley, CA (US); Alexandros HADJILAOU, Leipzig (DE); Angela Marie GREEN, Walnut Creek, CA (US); Maria Josefina COLOMA, Albany, CA (US)

(21) Appl. No.: 15/316,077

(22) PCT Filed: Oct. 30, 2015
(86) PCT No.: PCT/US2015/058281

§ 371 Date: Dec. 2, 2016

Related U.S. Application Data
(60) Provisional application No. 62/073,687, filed on Oct. 31, 2014.

(51) Int. Cl. G01N 33/68; G01N 33/58

(52) U.S. Cl. CPC G01N 33/6854; G01N 33/582; G01N 2333/185

(57) ABSTRACT

Methods of determining serotype specificity of an antibody that binds to a serotype of a pathogen are provided. Aspects of the methods include producing an assay mixture and evaluating the assay mixture for the presence of an antibody-pathogen-detectable label binding complex to determine the serotype specificity of the antibody. The assay mixture includes the antibody, at least first and second serotypes of the pathogen, a first detectable label that specifically binds to the first serotype of the pathogen, and a second detectable label that specifically binds to the second serotype of the pathogen. The methods find use in a variety of applications, such as for example determining the serotype specificity and/or cross reactivity of an antibody (e.g., an anti-dengue virus antibody).

### CROSS-REFERENCE TO RELATED APPLICATIONS

Pursuant to 35 U.S.C. §119 (e), this application claims priority to the filing date of U.S. Provisional Patent Application No. 62/073,687, filed Oct. 31, 2014; the disclosure of which application is herein incorporated by reference.

### INTRODUCTION

The 4 dengue virus serotypes (DENV1-4), members of the flavivirus genus, cause the most prevalent mosquito-borne viral illness in humans, with >3 billion people at risk for infection and up to 96 million cases annually worldwide. DENV infection can result in self-limiting dengue fever or more severe life-threatening disease. Severe disease is thought to be associated with the presence of serotype cross-reactive antibodies (Abs) that enhance viral uptake into target cells during a heterologous secondary infection. In contrast, type-specific Abs after a primary infection are important for homotypic protection. However, little is known about the protective role of cross-reactive Abs. To date, studies have analyzed DENV-specific B cells using ELISPOT assays and flow cytometry or by generating monoclonal Abs (MAbs) from memory B cells (MBCs) or plasma cells.

### SUMMARY

Methods of determining serotype specificity of an antibody that binds to a serotype of a pathogen are provided. Aspects of the methods include producing an assay mixture and evaluating the assay mixture for the presence of an antibody-pathogen-detectable label binding complex to determine the serotype specificity of the antibody. The assay mixture includes the antibody, at least first and second serotypes of the pathogen, a first detectable label that specifically binds to the first serotype of the pathogen, and a second detectable label that specifically binds to the second serotype of the pathogen. The methods find use in a variety of applications, such as for example determining the serotype specificity and/or cross reactivity of an antibody (e.g., an anti-dengue virus antibody).

### BRIEF DESCRIPTION OF THE FIGURES

FIG. 1 shows a schematic drawing of an assay according to embodiments of the present disclosure.

FIG. 2 shows graphs of the specificity of the detection of monoclonal antibodies according to embodiments of the present disclosure. Serotype-specific monoclonal antibodies targeting all four DENV serotypes were selected and conjugated to four distinct fluorescent dyes. The serotype specificity was confirmed by ELISA (upper row of graphs) and by flow cytometry (data not shown). The labeling efficiency was verified by flow cytometry (lower row of graphs).

FIG. 3—Four Nicaraguan DENV serotypes (DENV1-4) were propagated in C6/36 mosquito cells and concentrated. The concentrated DENV preps were further purified using discontinuous OptiPrep density centrifugation (FIG. 3, left panel). The purity was confirmed by SDS-PAGE/Coomassie blue (FIG. 3, right panel).

FIG. 4 shows a graph of pDENV dilutions vs. optical density (O.D.) as 562 nm. In order ensure equal protein concentration, the four purified DENV serotypes were standardized with a BCA assay.

FIG. 5A shows images of DENV serotype cross-reactive antibody secreting cells according to embodiments of the present disclosure. The detection of antibody-secreting cells using a DENV cross-reactive hybridoma and single serotype-specific monoclonal antibodies with all 4 DENV serotypes is shown. The serotype cross-reactive hybridoma, D11C, was plated in individual wells with equal numbers of cells for 48 hours. Antibody-secreting cells (ASC) were detected in each well by incubation with equal numbers of virions from all 4 DENV serotypes followed by incubation with a single DENV serotype-specific monoclonal antibody (MAb) in each well (n=3 wells/MAb). FIG. 5A depicts the visualization of each serotype-specific MAb by each filter. FIG. 5B shows a graph of the number of spots per well detected by each serotype-specific MAb (DENV1-4), the number of spots detected with all 4 serotype-specific MAbs, and total IgG. The graph indicates that equal numbers of spots were detected in total IgG, with all 4 DENV serotype-specific MAbs and each single serotype-specific MAb.

FIG. 6A shows the detection of antibody-secreting cells using a DENV cross-reactive hybridoma and a single DENV serotype with all 4 serotype-specific MAbs. DENV cross-reactive hybridoma D11C was plated in individual wells with equal numbers of cells per well for 48 hours. ASC were detected in each well by incubating with a single DENV serotype per set of experimental wells and visualized by incubation with all 4 serotype-specific MAbs (n=3 wells/condiQon). FIG. 6A indicates the detection of ASC when incubated with a single serotype and 4 serotype-specific MAbs. FIG. 6B shows a graph indicating the total number of spots per well from wells incubated with virions from DENV1-4 separately, wells incubated with all 4 DENV serotypes and all 4 antibodies, and total IgG. Equal numbers of spots were detected for total IgG, incubation with all 4 DENV serotypes, and incubation with single DENV serotypes.

FIG. 7 shows images indicating detection of ASCs from a DENV cross-reactive hybridoma by an assay according to the present disclosure. ASCs from DENV cross-reactive hybridoma D11C were detected by incubation with all 4 DENV serotypes and visualization with all 4 serotype-specific MAbs. The images from left to right show DENV1-4 filters separately and the merged filters from the same well.

FIG. 8 shows images indicating the detection of ASCs from a DENV serotype-specific hybridoma according to embodiments of the present disclosure. ASC from 2D22, a DENV2 serotype-specific hybridoma, were detected by incubation with all 4 DENV serotypes and visualization with all 4 serotype-specific MAbs. The images from left to right are DENV1-4 individual and merged filters.

FIG. 9 shows a graph indicating memory B cell activation from PBMCs. Memory B cell activation conditions: To determine which stimulation conditions would yield the most comprehensive activation profile for memory B cells, PMBCs from Nicaraguan blood donors were stimulated under the indicated conditions for 3 or 5 days: 1) IL-2/R848; 2) IL-21/CD40L; and 3) IL-21/CD40L/BAFF. Stimulated PBMCs were plated and assessed for the total number of ASC produced per 1×106 PBMCs. Stimulation patterns were assessed for 6 individual donors. IL-2/R848 for 5 days produced the most ASC per well.

FIG. 10 shows images of an assay performed on a DENV-immune donor from Nicaraguan National Blood Bank according to embodiments of the present disclosure. To visualize the memory B cell response, PBMCs were prepared from blood collected from an adult Nicaraguan blood donor. PBMCs were stimulated with IL-2 and R848 for 5 days. Stimulated cells were plated at an optimal dilution and visualized with all 4 DENV serotypes and 4 serotype-specific MAbs. FIG. 10 from left to right shows DENV1-4 separately and the merged filters from the same well.

FIG. 11 shows images of an assay performed on a convalescent sample from a Nicaraguan primary dengue case according to embodiments of the present disclosure. To visualize the memory B cell response, PBMCs collected during the convalescent phase (˜day 14 post-onset of illness) from an individual with a primary DENV3 infection were stimulated with IL-2 and R848 for 5 days. PBMCs were plated at optimal dilution and visualized with all 4 DENV serotypes and 4 serotype-specific MAbs. FIG. 11 from left to right show DENV1-4 filters separately and the merged filters from the same well.

FIG. 12 shows images of an assay performed on a convalescent sample from a Nicaraguan secondary dengue case according to embodiments of the present disclosure. PBMCs from an individual with secondary DENV infection were collected in the convalescent phase (˜14 days post-onset of illness) and stimulated with IL-2 and R848 for 5 days. PBMCs were plated at optimal dilution and visualized with all 4 DENV serotypes and 4 serotype-specific MAbs. FIG. 12 from left to right show DENV1-4 filters separately and the merged filters from the same well.

FIG. 13 shows images of an assay performed on an acute sample from a Nicaraguan secondary dengue case according to embodiments of the present disclosure. PBMCs from a patient with secondary DENV infection were collected during the acute phase of illness (day 4 post-onset of illness) and plated directly onto assay plates. Plasmablast responses were visualized by detection with all 4 DENV and all 4 serotype-specific MAbs. FIG. 13 from left to right show DENV1-4 filters separately and the merged filters from the same well.

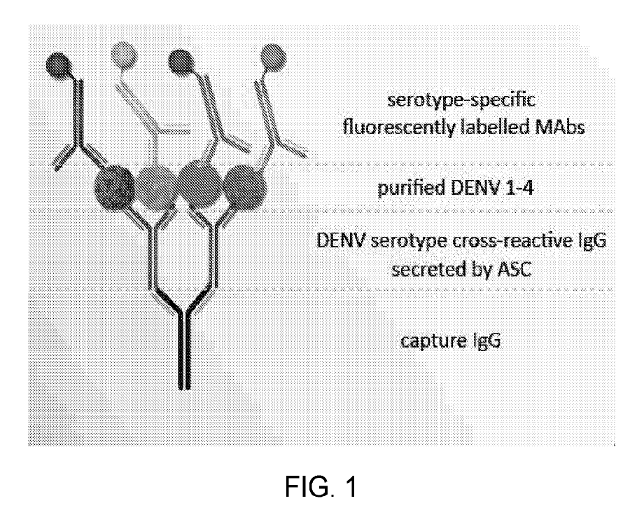

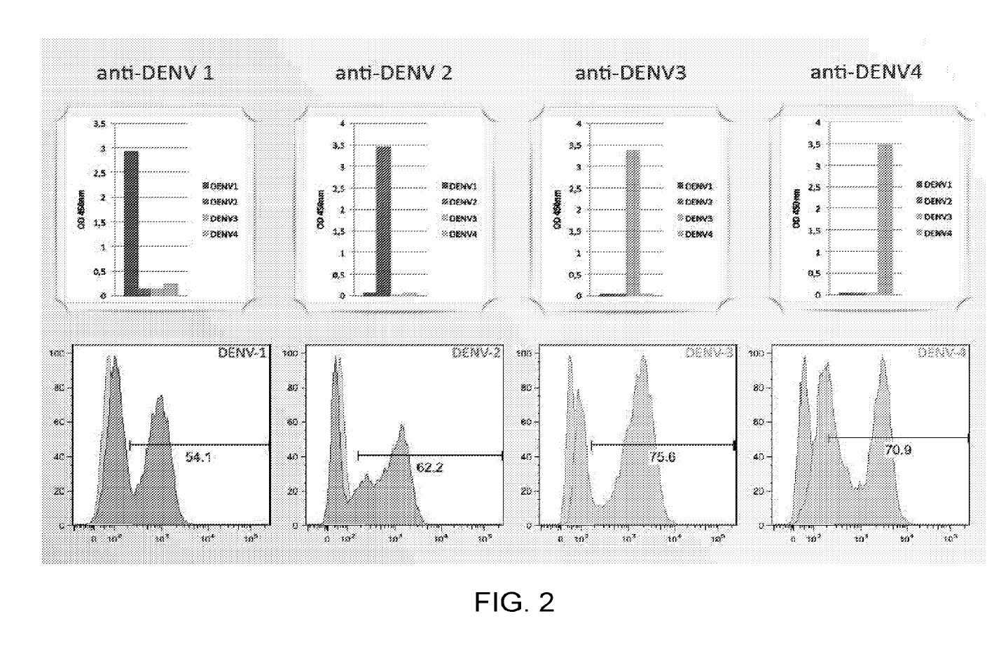

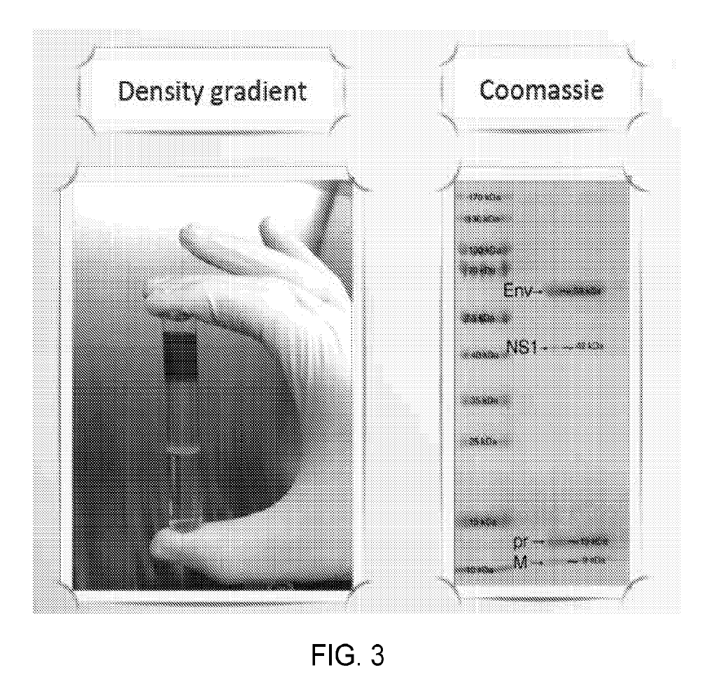

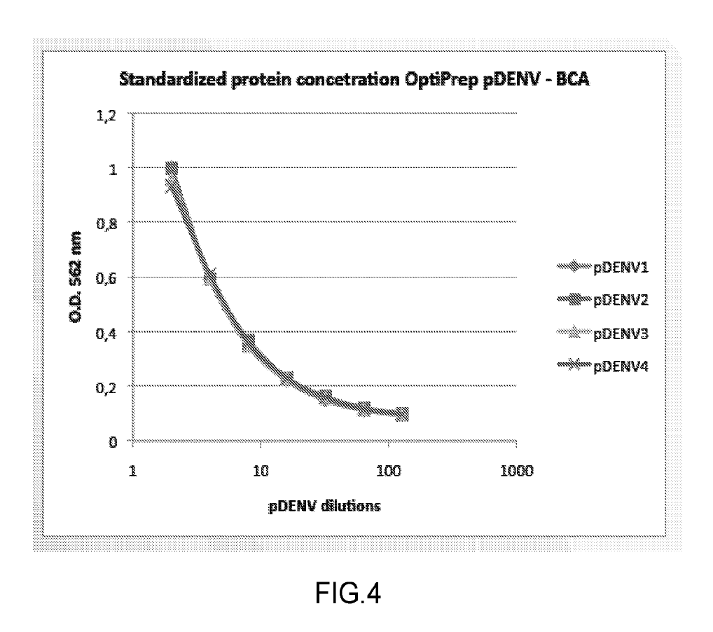

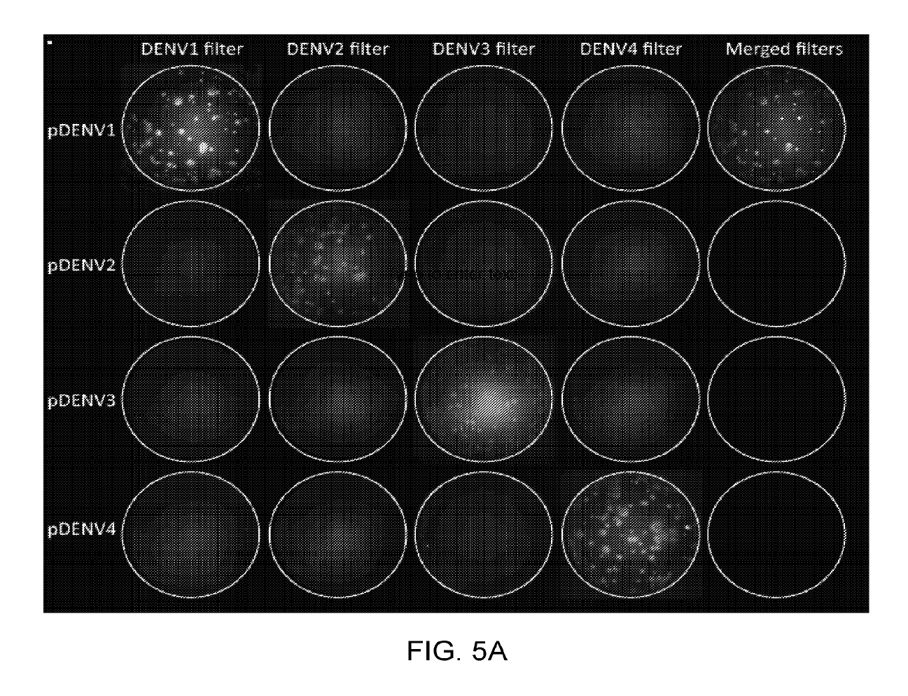

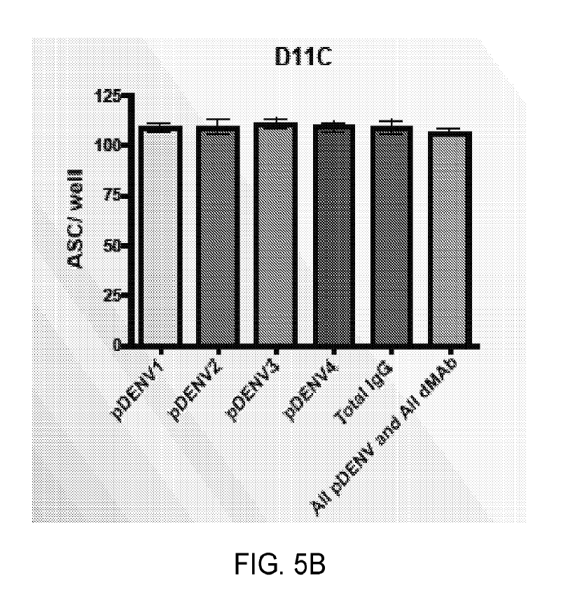

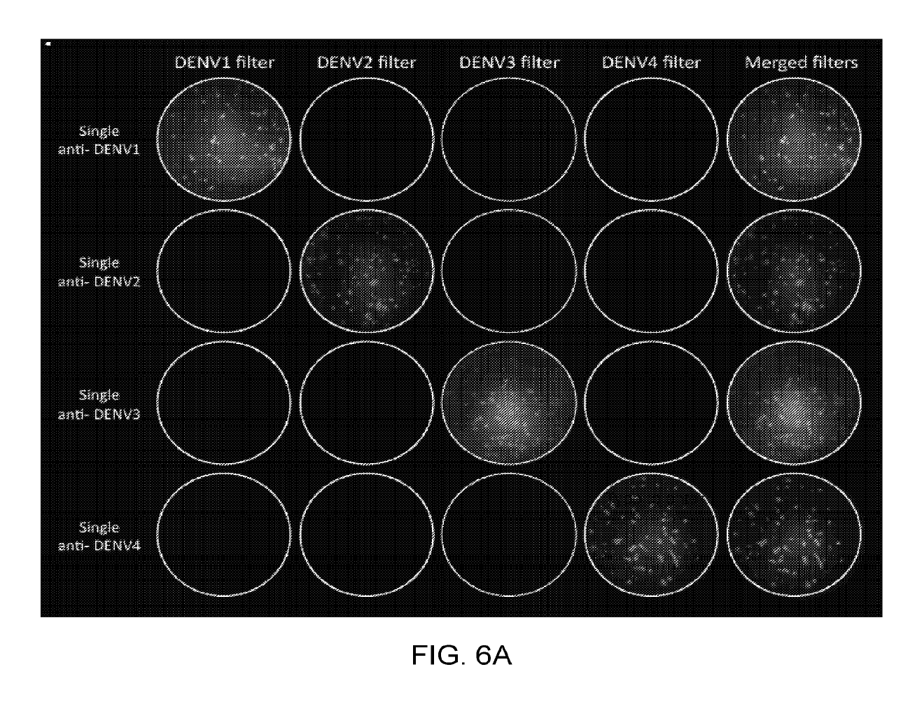

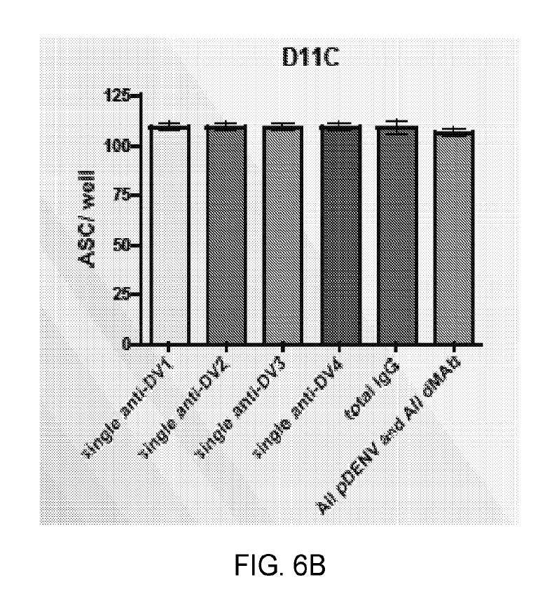

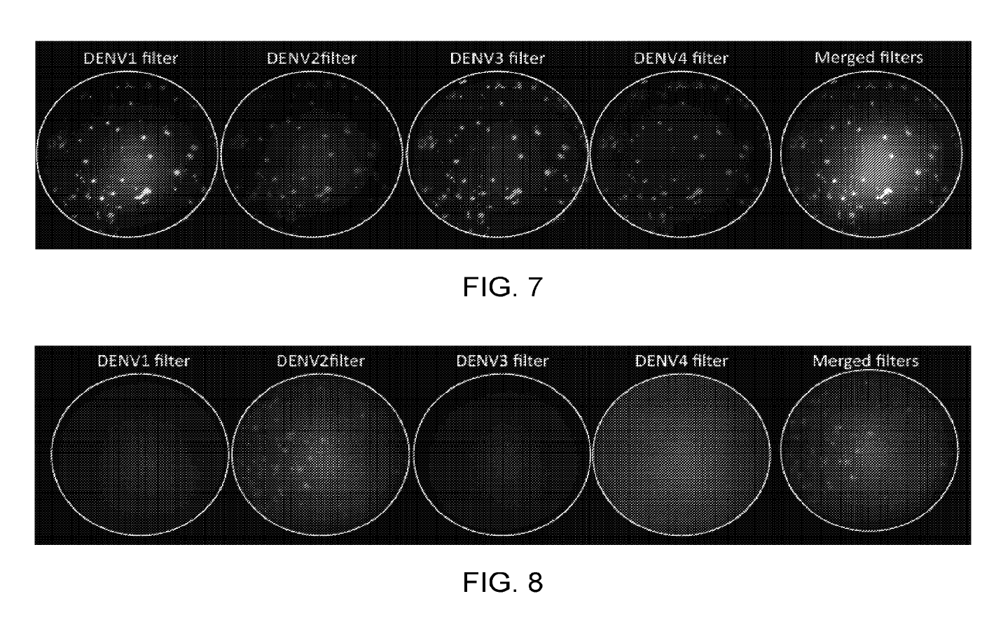

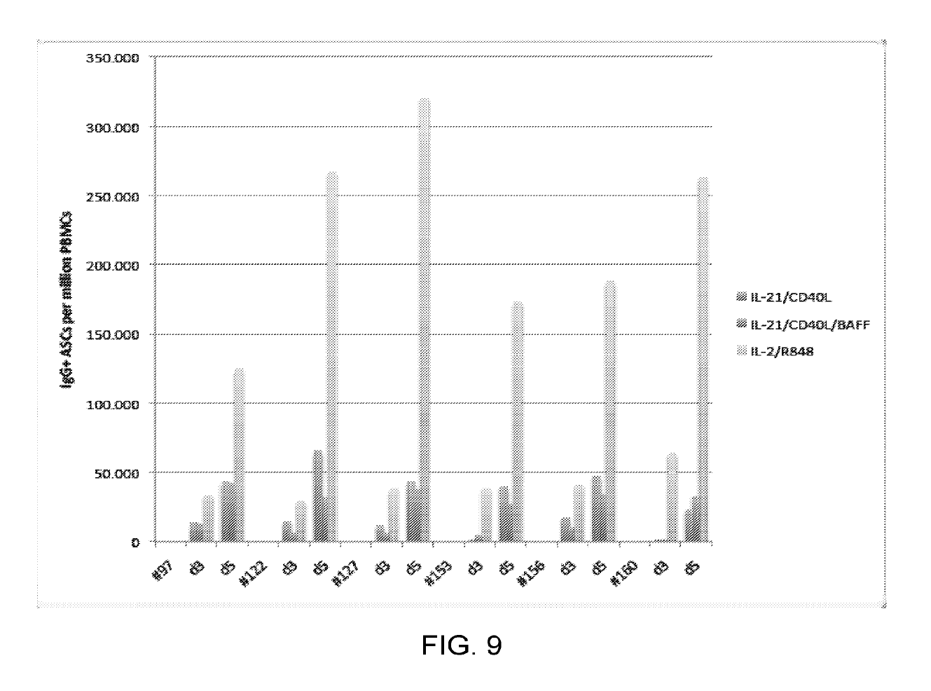

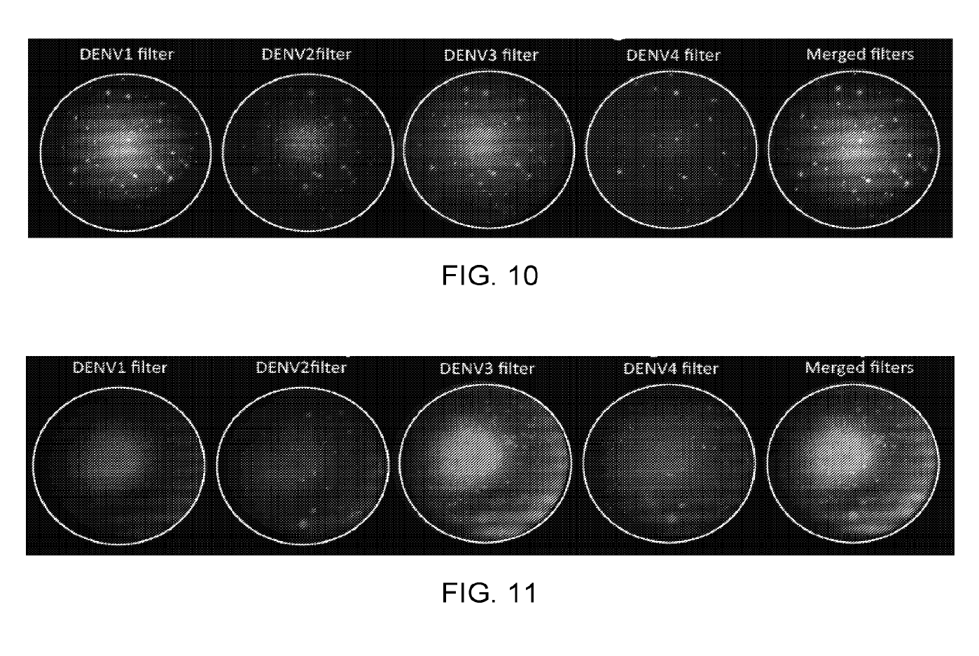

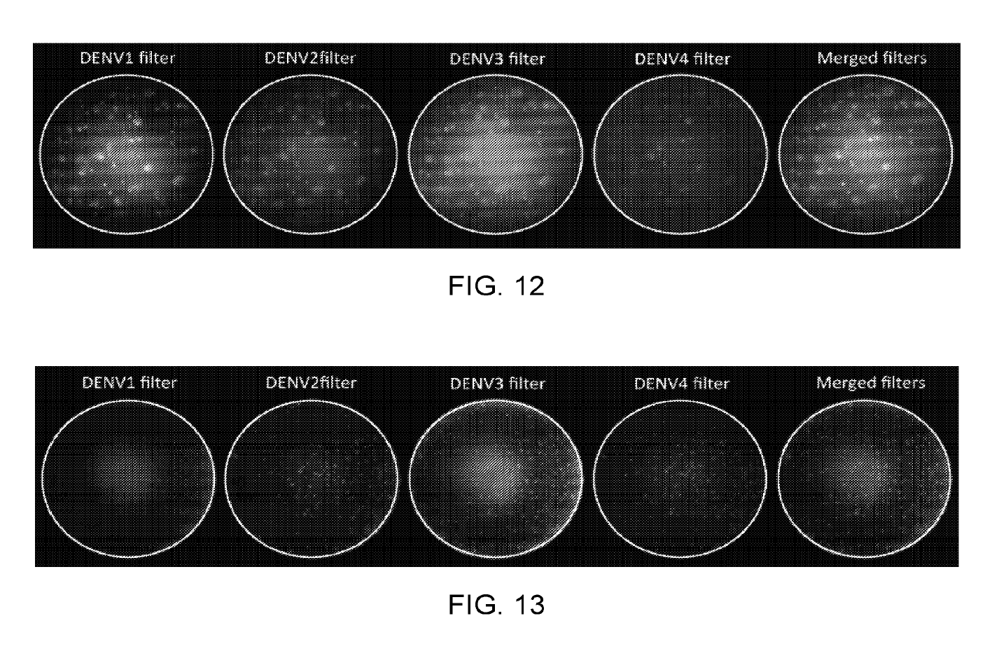

### DETAILED DESCRIPTION

Methods of determining serotype specificity of an antibody that binds to a serotype of a pathogen are provided. Aspects of the methods include producing an assay mixture and evaluating the assay mixture for the presence of an antibody-pathogen-detectable label binding complex to determine the serotype specificity of the antibody. The assay mixture includes the antibody, at least first and second serotypes of the pathogen, a first detectable label that specifically binds to the first serotype of the pathogen, and a second detectable label that specifically binds to the second serotype of the pathogen. The methods find use in a variety of applications, such as for example determining the serotype specificity and/or cross reactivity of an antibody (e.g., an anti-dengue virus antibody).

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Aspects of the present disclosure include a method of determining serotype specificity of an antibody that binds to a serotype of a pathogen. The term “serotype” is used in its conventional sense to refer to a category into a pathogen, e.g., a virus or bacterium, is placed based on its serological activity, e.g., in terms of the antigens it contains or the antibodies produced against it. The term pathogen is used broadly to refer to a variety of different types of pathogens, e.g., any disease-producing agent, such as a virus, bacterium, or other microorganism. Pathogens of interest therefore include viruses, e.g., Dengue viruses, bacterium, etc.

The terms “specificity,” “specific binding,” “specifically bind,” and the like, refer to the ability of the antibody to preferentially bind directly to the pathogen of interest relative to other entities, e.g., pathogens, molecules or moieties, in a solution or reaction mixture that may be present in a sample. In certain embodiments, the affinity between an antibody and the pathogen to which it specifically binds when they are specifically bound to each other in a binding complex is characterized by a K_{D} (dissociation constant) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. The number of different serotypes of the pathogen that the antibody may be tested for may vary, e.g., ranging in some instances from 2 to 10, such as 2 to 8, e.g., 2 to 6, including 3 to 5, e.g., 4.

The term “antibody” is used in the broadest sense and includes monoclonal antibodies (including full length monoclonal antibodies), polyclonal antibodies, and multispecific antibodies (e.g., bispecific antibodies), humanized antibodies, single-chain antibodies, chimeric antibodies, antibody fragments (e.g., Fab fragments), and the like. An antibody is capable of binding a target antigen. (Janeway, C., Travers, P., Walport, M., Shlomchik (2001) Immuno Biology, 5th Ed., Garland Publishing, New York). A target antigen can have one or more binding sites, also called epitopes, recognized by complementarity determining regions (CDRs) formed by one or more variable regions of an antibody. An immunoglobulin polypeptide immunoglobulin light or heavy chain variable region is composed of a framework region (FR) interrupted by three hypervariable regions, also called “complementarity determining regions” or “CDRs”. The extent of the framework region and CDRs have been defined (see, “Sequences of Proteins of Immunological Interest,” E. Kabat et al., U.S. Department of Health and Human Services, 1991). The framework region of an antibody, that is the combined framework regions of the constituent light and heavy chains, serves to position and align the CDRs. The CDRs are primarily responsible for binding to an epitope of an antigen. In the context of an Ig polypeptide, the term “constant region” is well understood in the art, and refers to a C-terminal region of an Ig heavy chain, or an Ig light chain. An Ig heavy chain constant region includes CH1, CH2, and CH3 domains (and CH4 domains, where the heavy chain is a p or an c heavy chain). In a native Ig heavy chain, the CH1, CH2, CH3 (and, if present, CH4) domains begin immediately after (C-terminal to) the heavy chain variable (VH) region, and are each from about 100 amino acids to about 130 amino acids in length. In a native Ig light chain, the constant region begins begin immediately after (C-terminal to) the light chain variable (VL) region, and is about 100 amino acids to 120 amino acids in length.

The method includes producing an assay mixture and evaluating the assay mixture for the presence of an antibody-pathogen-detectable label binding complex to determine the serotype specificity of the antibody. The assay mixture includes the antibody, e.g., as described above, at least first and second serotypes of the pathogen, a first detectable label that specifically binds to the first serotype of the pathogen, and a second detectable label that specifically binds to the second serotype of the pathogen. As reviewed above, the number of pathogen serotypes to be detected may vary. In some instances, the assay mixture further includes a third serotype of the pathogen and a fourth serotype of the pathogen.

The number of different detectable labels may be the same as the number of different target pathogen serotypes to be detected. As such, embodiments of the methods employ assay mixtures that include a first detectable label that specifically binds to the first serotype of the pathogen and a second detectable label that specifically binds to the second serotype of the pathogen. In some embodiments, the assay mixture includes a third detectable label that specifically binds to the third serotype of the pathogen, and a fourth detectable label that specifically binds to the fourth serotype of the pathogen. While the detectable labels may vary, in some embodiments, the first, second, third and fourth detectable labels each include a distinctly detectable label.

A detectable label may include a binding domain and a label domain. The terms “specific binding,” “specifically binds,” and the like, refer to the preferential binding of a domain (e.g., one binding pair member to the other binding pair member of the same binding pair) relative to other molecules or moieties in a solution or reaction mixture. The binding domain may specifically bind (e.g., covalently or non-covalently) to a particular epitope or narrow range of epitopes within the cell. In certain aspects, the binding domain non-covalently binds to a target. In such instances, the binding domain association with the binding target may be characterized by a KD (dissociation constant) of 10⁻⁵ M or less, 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less.

A variety of different types of binding domains may be employed. Binding domains of interest include, but are not limited to, antibody binding agents, proteins, peptides, haptens, nucleic acids, etc. The term “antibody binding agent” as used herein includes polyclonal or monoclonal antibodies or binding fragments thereof that are sufficient to bind to an analyte of interest. The binding fragments can be, for example, monomeric Fab fragments, monomeric Fab′ fragments, or dimeric F(ab)′2 fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

The label domain may be detectable based on, for example, fluorescence emission maxima, fluorescence polarization, fluorescence lifetime, light scatter, mass, molecular mass, or combinations thereof. In certain aspects, the label domain may be a fluorophore (i.e., a fluorescent label, fluorescent dye, etc.). Fluorophores can be selected from any of the many dyes suitable for use in analytical applications (e.g., flow cytometry, imaging, etc.). A large number of dyes are commercially available from a variety of sources, such as, for example, Molecular Probes (Eugene, Oreg.) and Exciton (Dayton, Ohio). Examples of fluorophores that may be incorporated into the microparticles include, but are not limited to, 4-acetamido-4′-isothiocyanatostilbene-2,2′disulfonic acid; acridine and derivatives such as acridine, acridine orange, acrindine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Green Fluorescent Protein (GFP); Reef Coral Fluorescent Protein (RCFP); Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; or combinations thereof. Other fluorophores or combinations thereof known to those skilled in the art may also be used. The fluorescent label may be distinguishable based on fluorescence emission maxima, and optionally further based on light scatter or extinction. In some embodiments, the distinctly detectable label is a fluorescent label.

In some embodiments, the methods include contacting the assay mixture, e.g., as described above, with a capture probe, e.g., so that the antibody is specifically bound to a capture probe, where the capture probe may be present on a surface of a substrate, i.e., solid support. A “capture probe” is an immobilized molecule that specifically binds to the antibody of interest. The terms “binds,” “binds to,” “binding,” and the like, as used herein (e.g., with reference to capture probes, binding pair members, and the like) refer to a non-covalent interaction between entities (e.g., between a metal ion affinity peptide and a metal ion, between an antibody and an antigen, and the like). The terms “specific binding,” “specifically binds,” and the like, refer to the preferential binding to a molecule (e.g., one binding pair member to the other binding pair member of the same binding pair, a capture probe to an analyte, a capture probe to a competitor, an immobilized control agent to a mobile control binding agent, etc.) relative to other molecules or moieties in a solution or reaction mixture. In some cases, “specifically binds” refers to preferential binding to one molecule in solution. In some cases, “specifically binds” refers to preferential binding to more than one molecule in solution (e.g., a member of a binding pair can specifically bind to two different molecules in the same solution).

“Immobilized” or “stably associated with the substrate” means that a molecule (e.g., capture probe, immobilized control agent, etc.) and the substrate maintain their position relative to each other in space under the conditions of use, e.g., under the assay conditions. As such, an immobilized capture probe and the substrate can be non-covalently or covalently stably associated with each other. Examples of non-covalent association include non-specific adsorption, binding based on electrostatic (e.g., ion-ion pair interactions), Van der Waals forces, hydrophobic interactions, hydrogen bonding interactions, and the like. Examples of covalent binding include covalent bonds formed between a capture probe and a functional group present on the substrate.

As mentioned above, a “capture probe,” also referred to herein as an “immobilized capture probe,” is an immobilized molecule that specifically binds to the antibody of interest. In some embodiments, the affinity between a capture probe and the molecule to which it specifically binds when they are specifically bound to each other in a binding complex is characterized by a K_{D} (dissociation constant) of 10⁻⁵ M or less, 10⁻⁶ M or less, such as 10⁻⁷ M or less, including 10⁻⁵ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less. “Affinity” refers to the strength of binding, increased binding affinity being correlated with a lower Kd.

A variety of different types of specific binding agents may be employed as a capture probe. A capture probe is therefore considered to include a binding pair member (defined below). Specific binding agents that can be used as a capture probe include antibody binding agents, proteins, peptides (e.g., glutathione, epitopes, tags, etc.), haptens, nucleic acids, metal ions (e.g., Ni⁺², Co⁺², Fe⁺³, Al⁺³, Zn⁺², Cu⁺², etc.), carbohydrates (e.g., amylose, maltose), and the like. The term “antibody binding agent” as used herein includes polyclonal or monoclonal antibodies or fragments that are sufficient to bind to an analyte of interest. The antibody fragments can be, for example, monomeric Fab fragments, monomeric Fab′ fragments, or dimeric F(ab)′₂ fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

A “binding pair member” is one of a first and a second moiety, wherein the first and the second moiety have a specific binding affinity for each other. Together the first and second moiety can be referred to as a “binding pair,” and each moiety (first and second) of the binding pair is therefore a binding pair member. Accordingly, a molecule may be said to include a binding pair member. A molecule may also be said to include two or more binding pair members, each of which can be members of different binding pairs. As mentioned above, in some instances the affinity of a first binding pair member to a second binding pair member of a give binding pair is characterized by a K_{D} (dissociation constant) of 10⁻⁵ M or less, e.g., 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less. “Affinity” refers to the strength of binding, increased binding affinity being correlated with a lower Kd.

The substrate, i.e., solid support to which the capture probe is immobilized in these embodiments may vary. Substrates may include any convenient material, where materials of interest include polymeric materials, e.g., plastics, glasses, etc.

In some embodiments, the method includes washing unbound detectable label from the binding complex. As such, a washing step may be performed, as desired, e.g., to remove any unbound detectable labels and other sample components. Washing may be performed using any convenient protocol, such as by contacting the substrate surface with a suitable wash buffer (e.g., PBS, HEPES) and separating the surface from the wash fluid. A given washing protocol may include one or more distinct washing steps, as desired.

In some embodiments, the method includes determining a cross reactivity of the antibody to different serotypes of the pathogen. As such, the methods include identifying the label(s) present at the capture probe sight and, based on the identified labels, determining any immunologic reaction between two or more pathogen(s) and the antibody that is being evaluated. In some embodiments, the evaluating includes evaluating the assay mixture for the presence of each of the distinctly detectable labels.

In some embodiments, the method includes producing the antibody. The antibody may be produced using any convenient protocol, e.g., as described below. In some embodiments, producing the antibody includes providing an inducible cell and inducing the cell to produce the antibody. In some embodiments, the inducible cell is obtained from a blood sample of a subject exposed to the pathogen.

Aspects of the present disclosure include a method of determining serotype specificity of an anti-dengue virus antibody that binds to a serotype of a dengue virus. The method includes specifically binding the anti-dengue virus antibody to a capture probe on a surface of a substrate to produce an anti-dengue virus antibody-capture probe complex, contacting the anti-dengue virus antibody-capture probe complex with an assay mixture, and evaluating the assay mixture for the presence of an anti-dengue virus antibody-dengue virus-detectable label binding complex to determine the serotype specificity of the anti-dengue virus antibody. The assay mixture includes a first, second, third and fourth serotype of the dengue virus, a first detectable label that specifically binds to the first serotype of the dengue virus, a second detectable label that specifically binds to the second serotype of the dengue virus, a third detectable label that specifically binds to the third serotype of the dengue virus, and a fourth detectable label that specifically binds to the fourth serotype of the dengue virus.

In some embodiments, the method includes determining a cross reactivity of the anti-dengue virus antibody to different serotypes of the dengue virus. In some embodiments, the method includes producing the anti-dengue virus antibody. In some embodiments, producing the anti-dengue virus antibody includes providing an inducible cell and inducing the cell to produce the anti-dengue virus antibody. In some embodiments, the inducible cell is obtained from a blood sample of a subject exposed to the dengue virus.

### Utility

The subject methods find use in a variety of different applications where determination of serotype specificity and/or cross-reactivity of an antibody that binds to a serotype of a pathogen is desired. The assay can be used to screen and analyze the B cell population of DENV-infected and/or vaccinated individuals. The technique/approach can be applied to any other pathogen with different serotypes/subtypes and therefore has applications beyond dengue virus.

The subject methods find use in detecting serotype-specific and cross-reactive antibody-secreting cells and characterizing multiple-antigen specificity on a per-cell basis. For example, this can be applied to dengue virus, as well as for many other pathogens, or even applied to numerous other antigens for determination of B cell antibody specificity.

The subject methods find use in the determination of serotype specificity and/or cross-reactivity of an antibody on a single cell basis. In addition, The subject methods provide for minimal manipulation of B cells and thus may be more representative of a cells in their typical in vivo environment.

### Kits

Aspects of embodiments of the present disclosure further include kits configured for use in the methods described herein. In some instances, the kits include at least first and second serotypes of a pathogen, a first detectable label that specifically binds to the first serotype of the pathogen, and a second detectable label that specifically binds to the second serotype of the pathogen.

In some embodiments, the kit includes a third serotype of the pathogen and a fourth serotype of the pathogen.

In some embodiments, the kit includes a third detectable label that specifically binds to the third serotype of the pathogen, and a fourth detectable label that specifically binds to the fourth serotype of the pathogen.

In some embodiments, the kit includes a substrate having a capture probe on a surface thereof, wherein the capture probe is configured to specifically bind to an antibody that binds to a serotype of the pathogen.

In some embodiments, the capture probe is configured to specifically bind to a constant region of the antibody.

In certain embodiments, the kit includes a packaging configured to contain the components of the kit. The packaging may be a sealed packaging, such as a sterile sealed packaging. By “sterile” is meant that there are substantially no microbes (such as fungi, bacteria, viruses, spore forms, etc.). In some instances, the packaging may be configured to be sealed, e.g., a water vapor-resistant packaging, optionally under an air-tight and/or vacuum seal.

The kits may further include additional reagents, such as but not limited to, buffers, e.g., assay buffer, wash buffer, etc.

In addition to the above components, the subject kits may further include instructions for practicing the subject methods. These instructions may be present in the subject kits in a variety of forms, one or more of which may be present in the kit. One form in which these instructions may be present is as printed information on a suitable medium, e.g., a piece or pieces of paper on which the information is printed, in the packaging of the kit, in a package insert, etc. Another means would be a computer readable medium, e.g., portable flash drive, CD, DVD, Blu-Ray, computer-readable memory, etc., on which the information has been recorded or stored. Yet another means that may be present is a website address which may be used via the Internet to access the information at a removed site. Any convenient means may be present in the kits.

As can be appreciated from the disclosure provided above, embodiments of the present invention have a wide variety of applications. Accordingly, the examples presented herein are offered for illustration purposes and are not intended to be construed as a limitation on the invention in any way. Those of ordinary skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by mass, molecular mass is mass average molecular mass, temperature is in degrees Celsius, and pressure is at or near atmospheric.

### EXAMPLES

### I. Detection of B Cell Antigen Specificity to 4 Different Dengue Virus Serotypes (DENV1-4)

### A. Results and Discussion

Embodiments of the invention include an assay that allows the detection of B cell antigen specificity to four different pathogen serotypes (e.g., 4 different dengue virus serotypes (DENV1-4)) on a per-cell basis. This technique allows visualization of serotype cross-reactivity versus type-specificity per cell. Thus, this technique enables quantification of the breadth, magnitude, specificity and cross-reactivity of B cell responses following sequential infections (e.g., sequential DENV infections). The assay of these embodiments provides for detection of B cell antigen-specificity to four serotypes (e.g., the four DENV serotypes) in a single well on a per-cell basis using four distinct fluorescent dyes. Memory B cells are stimulated in vitro to differentiate them into antibody-secreting cells, or plasmablasts/plasma cells from the acute phase of infection are examined directly. Serial dilutions of the antibody-secreting cells (ASCs) are incubated in plates coated with an Fc-directed antibody (anti-Fcγ, Fcα or Fcμ) to capture the secreted antibodies, which are then incubated with purified virions (e.g., DENV virions) from four serotypes (e.g., the four DENV serotypes). Visualization is achieved using four serotype-specific antibodies (e.g., mouse DENV serotype-specific monoclonal antibodies (MAbs)) that are labeled with different fluorophores, and detection is performed using an fluorescent reader. The assay has been validated with both peripheral blood mononuclear cells from human DENV-immune individuals as well as serotype-specific and cross-reactive human MAb hybridomas.

For example, embodiments of the invention provide a novel approach to determine the frequency of serotype-specific and serotype cross-reactive MBC responses on a per-cell basis. MBC specificity to all 4 DENV serotypes can be evaluated simultaneously in individual cells. The MBCs from cryopreserved peripheral blood mononuclear cells (PBMCs) of DENV-exposed individuals were stimulated in vitro for 3-5 days to become Ab-secreting cells and then incubated in ELISPOT plates coated with a human IgG capture Ab for 1-2 days. Subsequently, OptiPrep-purified virions from the four serotypes (Nicaraguan DENV1-4) were added to bind to DENV serotype-specific and cross-reactive Abs secreted by the Ab-secreting B cells. Visualization was achieved using four mouse anti-E Domain III serotype-specific MAbs that were labeled with different fluorophores. Serotype-specific MBCs and MBCs cross-reactive to 2, 3, or 4 DENV serotypes were detected in different individuals, with type-specific and cross-reactive MAb hybridomas used as controls. This method is employed to analyze longitudinal samples (acute, convalescent, and 3, 6, 12, and 18 months post-illness) from primary and secondary DENV infections in a hospital-based dengue study in Nicaragua. This new technique will further the understanding of how MBC specificity evolves over time, how the serotype-specific and cross-reactive responses associate with disease outcome, and how serial infections with heterotypic serotypes drive MBC specificity.

To date, one main approach to analyze and characterize the serotype cross-reactivity of the B cell population in dengue virus (DENV)—infected individuals is the generation of monoclonal antibodies that can be further characterized in various immunological assays, including the characterization of their serotype reactivity. Human hybridoma production is laborious and includes the transformation of B cells with Epstein Barr Virus (EBV). The efficiency of B cell transformation is low (3-10%), which could potentially lead to a significant loss of B cells necessary for a representative and comprehensive characterization of the entire B cell population that recognizes DENV. Another approach is the traditional enzymatic B cell ELISPOT assay, which thus far has been limited to enumerating the antibody—secreting cells (ASCs) reacting to one antigen. The use of fluorescent dyes for detection of DENV-specific antibodies secreted by B cells instead of using an enzymatic reaction to develop the B cell antibody spots increases the sensitivity and reduces the background, which results from non-specific staining during the enzymatic reaction. In addition, the use of fluorescent dyes enables the characterization of the cross-reactivity of an antibody secreted by a B cell to more than two antigens (in our case four different DENV serotypes). The assay includes the capture of antibodies secreted by B cells on a plate followed by detection of antigen specificity, which enables determination of serotype specificity on a per-cell basis, rather than only at the cell population level as in the conventional enzymatic ELISPOT.

The assay can be used to identify serotype-specific and cross-reactive B cells in DENV-infected individuals as well in vaccines. The assay can be used to test the efficiency of a vaccine in generating a full B cell response against the DENV envelope glycoprotein and to compare the cross-reactivity of the B cell population between individuals with naturally acquired dengue immunity and recipients of candidate dengue vaccines.

The assay may also be used for the detection of different isotypes of immunoglobulins (IgG, IgM, IgA) directed against the envelope of DENV. Besides quantification of DENV-specific IgG antibodies, which is the immunoglobulin isotype that most of the research has focused on, the assay can be used to detect IgM and IgA antibody-secreting cells (ASCs) in DENV-infected individuals.

Other embodiments may include mutant viruses during the detection procedure to further dissect the specificity of the B cell antibody response; for instance, viruses have been generated that are ablated for a particular antibody epitope or that contain a serotype-specific epitope transplanted from a different serotype, and these could be used in the assay.

Additionally, there are two different types of B cell assays, the ex vivo and the in vitro assay. While the ex vivo assay is used to detect plasmablasts (PBs)/plasma cells (PCs) directly in the peripheral blood mononuclear cells (PBMCs) from individuals under study, the in vitro assay requires activation of the memory B cells (MBCs) in the PBMCs using various cytokines and TLR agonists for stimulation. Thus, other embodiments of the assay include the detection of different types of antibody-secreting cells (ASCs), such as PBs/PCs or MBCs. Further, the use of different combinations of these stimulants can result in activation of different subsets of MBCs.

The subject method can be used to analyze B cell hybridomas that secrete monoclonal antibodies of different type-specificity and to determine the relative affinity of the monoclonal antibodies to virions of the four different DENV serotypes or, in other applications, the relative affinity of monoclonal antibodies to distinct variants of the antigen in question.

Embodiments of the invention include a new assay that allows visualization of DENV serotype-specific and cross-reactive antibody responses on a per-cell basis. Detection of ASC was the same when incubated with each serotype separately and 4 serotype-specific MAbs, 4 DENV serotypes and each serotype-specific MAbs separately, or all 4 DENV serotypes and 4 serotype-specific MAbs, indicating steric hindrance and/or competition among serotypes or MAbs was not an issue when multiple viruses and antibodies were used in combination for detection. Optimal memory B cell stimulation was achieved when PBMCs were stimulated in the presence of IL-2 and R848 for 5 days prior to the assay. The assay was able to visualize not only memory B cell responses in blood bank donors and the convalescent phase of DENV infection in the context of a hospital-based dengue study in Nicaragua, but also plasmablast responses during acute DENV infection.

### B. Materials and Methods

Context

| | |
|---|---|
| p1-2 | Materials |
| p2-7 | Experimental procedure |
| p3-4 | DAY 1: PBMC activation |
| p3 | PART A: thawing the cells |
| p4 | PART B: activating the cells |
| p4-5 | DAY 4 or 5: Preparing the 96-well FluoroSpot plate |
| p4 | PART A: Coating the plate |
| p5 | PART B: Blocking the plate |
| p5-6 | DAY 5: Plating the cells in the 96-well FluoroSpot |
| | plate |
| p5 | PART A: PBMC preparation |
| p6 | PART B: seeding the activated & washed |
| | PBMCs |
| p6-7 | DAY 7: Developing the FluoroSpot plate |
| p6 | PART A: Antigen incubation |
| p7 | PART B: Blocking the capture antibody |
| p7 | PART C: Detection MAbs incubation |

Materials:
Sterile TC Hood
37° C., 5% CO₂ cell culture incubator
sterile 50 mL conical tubes
sterile 1.5 mL Eppendorf tubes
500 μl amicon tubes—50 kDa cut-off membrane
RPMI supplemented with:
   10% FBS
   1% GlutaMax
   1% Hepes
   1% P/S
30 μl DNaseI aliquot—in −20° C. box at 10 U/μl stock concentration
24-well; Standard tissue culture; Flat-bottom; Growth area: 2 cm²; Well volume: 3.5 mL—Catalog #08-772-1 (Corning No.: 353047)
IL-2 in 1000 U/μL stock concentration at −20° C. for frequent use or −80° C. for long storage
R848 in 1 mg/mL stock concentration at −20° C.—from Invirogen
96-well FluoroSpot plate (Mscrn HTS IP-FL 0.45 UM CLEAR, Millipore Catalog # S5EJ104I07)
70% EtOH
sterile 1×PBS
PBS-Tween 0.05% (10×PBS diluted to 1×+500 μl of Tween in 1 L of PBS)
OptiPrep® purified DENV serotypes
sterile reservoir/pippette basin (Denville PS, 25 ml Catalog # P8825-S)
300P 12-well multichannel pippette
cover for unused wells
anti-human IgG capture antibody (Fc-specific)
human serum immunoglobulin
Four detection monoclonals

| MAb | Description | Designated Qdot | Filter # on reader |
|---|---|---|---|
| E95 | mouse anti-DENV1 | Qdot ® 565 | 4 |
| E96 | mouse anti-DENV2 | Qdot ® 705 | 5 |
| 5J7 | human anti-DENV3 | Qdot ® 625 | 6 |
| E88 | mouse anti-DENV4 | Qdot ® 525 | 3 |

Experimental Procedure

### Day 1: PBMC Activation

Part A: Thawing the Cells
Select the sample to activate.
Before you thaw the sample make sure you warm up (37° C.) and prepared 30 mL of PBMC-thaw-media (RPMI 10% FBS, 1% HEPES, 1% P/S, 1% GlutaMax and a 30 μl DNaseI aliquot)
Take the sample from Liquid Nitrogen (LiNi) and thaw it quick (2-3 min) in the 37° C. water bath.
Transfer the 250 μl-1 mL sample with a 1000 μl pipette to a 50 mL conical tube.
Tilt the conical tube to about 45° and slowly add the PBMC-thaw-media while you keep a constant slow rotation of the tube.
Seal and label the tube.
Centrifuge for 5 min at 1500 rpm
   Note: this can be performed either at RT or 4° C.
Discard the supernatant and resuspend the cells in 1 mL fresh RPMI
Take 10 μl of the cells and determine the cell concentration with the hemocytometer

Part B: Activating the Cells
Once the cell concentration is known, dilute or concentrate the cells to 1×10⁶-2×10⁶ cells/mL of media.
Add 1 mL of the cells in one well of a 24-well plate.
Observe the cell density under the microscope to assure sufficient cell confluency.
Prepare IL-2+R848 working concentration. For the activation of PBMCs you need 1000U of IL-2 and 2.5 μg of R848 per mL of activation media.
Label the wells and the plate and place the plate in the 37° C. for 5 days.

### Day 4 or 5: Preparing the 96-Well FluoroSpot Plate

Part A: Coating the Plate
Take a new sterile 96-well IP-FL plate and calculate how many wells of the plate you will need for your experiment. Use the white tape to seal the wells you don't need to use, for future experiments.
Using the multichannel pipette add 50 μl/well of 70% EtOH for 2 min at RT.
Discard the EtOH and wash 3 times with sterile 1×PBS
Coat the plate with 2 μg/well of the capture anti-human IgG (Fc fragment specific) in 50 μl final volume/well.
Incubate at 4° C. overnight (>8-10 hours) or at 37° C. for two hours.

### Part B: Blocking the Plate

Discard the supernatant
Wash 3× with 1× sterile PBS
Block the plate with 200 μl/well of complete media (supplemented RPMI)
Incubate at 4° C. overnight (>8-10 hours) or at 37° C. for two hours.

### Day 5: Plating the Cells in the 96-Well FluoroSpot Plate

Part A: PBMC Preparation
Transfer the 1 mL of the activated cells in 1.5 mL Eppendorf tube using a 1000 pipette
Centrifuge at 1500 rpm for 5 min
Transfer the supernatant in a new, clean and sterile 1.5 mL Eppendorf tube. This is the activation supernatant that contains the antibodies secreted by the memory b cells.
Resuspend the cell pellet in 1.3 mL of fresh media (supplemented RPMI)
Take 10 μl of the cells and determine the cell concentration with the hemocytometer
Centrifuge the resuspended pellet at 1500 rpm for 5 min
Discard the supernatant this time and resuspend the cell pellet in 1.3 mL of fresh media (supplemented RPMI)
Centrifuge at 1500 rpm for 5 min
After those two washes your cells are ready to be seeded to the FluoroSpot plate.

### Extra Step: Concentration of the Activation Sup for Neutralization

To concentrate the activation sup. use the 0.5 mL Amicon microcentrifuge filters (50 KDa). Since you have 1 mL you will have two spins for each sample.
Add 500 μl of the activation sup to the amicon filter and spin at maximum speed for 3-4 min.

### Part B: Seeding the Activated & Washed PBMCs

Resuspend the pellet in 200 μl of fresh media (approximately 1×10⁶-2×10⁶ cells should be seeded in the first dilution)

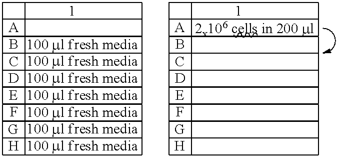

Add 100 μl of fresh media in all 7 wells after the first well to make the serial 1:2 dilution
Plate the 200 μl containing the cells in the first well. See diagram above for details.
Add fresh media to the wellst hat have only 100 μl to reach a final volume of 200 μl.
Label and incubate at 37° C., 5% CO₂ for 48 hours

### Day 7: Developing the FluoroSpot Plate

Part A: Antigen Incubation
Discard the cells and the media
Wash the plate 3 times with 250 μl/well of 1×PBS-Tween 0.05%
At second wash, leave the PBS-T for 5-10 min
In the meanwhile get the purified virus from −80° C. and calculate the amount of virus you will need
Dilute each purified DENV serotype to 20 μg/mL. The final volume is 50 μl/well
Incubate for 1 h at 37° C.
Wash 3× with 250 μl/well of PBS-T

### Part B: Blocking the Capture Antibody

Dilute the human gamma globulin 1:500 in PBS
Incubate 50 μl/well for 1 h at 37° C.
Wash 3× with 250 μl/well of PBS-T

### Part C: Detection MAbs Incubation

Pippette 1 mL PBS in an 1.5 mL Eppendorf tube
Add all the four detection MAbs in a 1:1000 dilution. If you need for example final volume of 5 mL (entire plate), add 5 μl of each detection MAb to the 1 mL of PBS.
Spin at maximum speed for 5 min
Transfer 950 μl of the supernatant to a new 1.5 mL Eppendorf tube
Spin again at maximum speed for 5 min
Transfer 950 μl to the remaining 4 mL of PBS (if you are need a total of 5 mL. Just an example)
Incubate 50 μl/well for 1 h at 37° C.
Wash 3× with 250 μl/well of PBS-T
Remove the plastic cover
Add 200 μl/well of ddH₂O and use the vaccuum manifold
If neccesary use the airflow of a TC hood to fast dry the plate.
Read on the FluoroSpot reader.

Notwithstanding the appended clauses, the disclosure set forth herein is also defined by the following clauses:

1. A method of determining serotype specificity of an antibody that binds to a serotype of a pathogen, the method comprising:

producing an assay mixture comprising:
the antibody;
at least first and second serotypes of the pathogen;
a first detectable label that specifically binds to the first serotype of the pathogen; and
a second detectable label that specifically binds to the second serotype of the pathogen; and

evaluating the assay mixture for the presence of an antibody-pathogen-detectable label binding complex to determine the serotype specificity of the antibody.

2. The method of Clause 1, further comprising determining a cross reactivity of the antibody to different serotypes of the pathogen.
3. The method of Clauses 1 or 2, further comprising producing the antibody.
4. The method of Clause 3, wherein the producing comprises providing an inducible cell and inducing the cell to produce the antibody.
5. The method of Clause 4, wherein the inducible cell is obtained from a blood sample of a subject exposed to the pathogen.
6. The method of any of Clauses 1 to 5, wherein the assay mixture further comprises a third serotype of the pathogen and a fourth serotype of the pathogen.
7. The method of Clause 6, wherein the assay mixture further comprises a third detectable label that specifically binds to the third serotype of the pathogen, and a fourth detectable label that specifically binds to the fourth serotype of the pathogen.
8. The method of Clause 7, wherein the first, second, third and fourth detectable labels each comprise a distinctly detectable label.
9. The method of Clause 8, wherein the evaluating comprises evaluating the assay mixture for the presence of each of the distinctly detectable labels.
10. The method of Clauses 8 or 9, wherein the distinctly detectable label is a fluorescent label.
11. The method of any of Clauses 1 to 10, wherein the pathogen comprises a virus.
12. The method of Clause 11, wherein the virus is a dengue virus.
13. The method of any of Clauses 1 to 10, wherein the pathogen comprises a bacterium.
14. The method of any of Clauses 1 to 13, wherein the antibody is specifically bound to a capture probe.
15. The method of Clause 14, wherein the capture probe is present on a surface of a substrate.
16. The method of any of Clauses 1 to 15, further comprising washing unbound detectable label from the binding complex.
17. A method of determining serotype specificity of an anti-dengue virus antibody that binds to a serotype of a dengue virus, the method comprising:

specifically binding the anti-dengue virus antibody to a capture probe on a surface of a substrate to produce an anti-dengue virus antibody-capture probe complex;

contacting the anti-dengue virus antibody-capture probe complex with an assay mixture comprising:
a first, second, third and fourth serotype of the dengue virus;
a first detectable label that specifically binds to the first serotype of the dengue virus;
a second detectable label that specifically binds to the second serotype of the dengue virus;
a third detectable label that specifically binds to the third serotype of the dengue virus; and
a fourth detectable label that specifically binds to the fourth serotype of the dengue virus;

evaluating the assay mixture for the presence of an anti-dengue virus antibody-dengue virus-detectable label binding complex to determine the serotype specificity of the anti-dengue virus antibody.

18. The method of Clause 17, further comprising determining a cross reactivity of the anti-dengue virus antibody to different serotypes of the dengue virus.
19. The method of any of Clauses 17 or 18, further comprising producing the anti-dengue virus antibody.
20. The method of Clause 19, wherein the producing comprises providing an inducible cell and inducing the cell to produce the anti-dengue virus antibody.
21. The method of Clause 20, wherein the inducible cell is obtained from a blood sample of a subject exposed to the dengue virus.
22. A kit comprising:

at least first and second serotypes of a pathogen;

a first detectable label that specifically binds to the first serotype of the pathogen; and

a second detectable label that specifically binds to the second serotype of the pathogen.

23. The kit of Clause 22, further comprising a third serotype of the pathogen and a fourth serotype of the pathogen.
24. The kit of Clause 23, further comprising a third detectable label that specifically binds to the third serotype of the pathogen, and a fourth detectable label that specifically binds to the fourth serotype of the pathogen.
25. The kit of any of Clauses 22 to 24, further comprising a substrate having a capture probe on a surface thereof, wherein the capture probe is configured to specifically bind to an antibody that binds to a serotype of the pathogen.
26. The kit of Clause 25, wherein the capture probe is configured to specifically bind to a constant region of the antibody.

Although the foregoing invention has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of determining serotype specificity of an antibody that binds to a serotype of a pathogen, the method comprising:
producing an assay mixture comprising:
the antibody;
at least first and second serotypes of the pathogen;
a first detectable label that specifically binds to the first serotype of the pathogen; and
a second detectable label that specifically binds to the second serotype of the pathogen; and
evaluating the assay mixture for the presence of an antibody-pathogen-detectable label binding complex to determine the serotype specificity of the antibody.

2. The method of claim 1, further comprising determining a cross reactivity of the antibody to different serotypes of the pathogen.

3. The method of claim 1, further comprising producing the antibody.

4. The method of claim 3, wherein the producing comprises providing an inducible cell and inducing the cell to produce the antibody.

5. The method of claim 4, wherein the inducible cell is obtained from a blood sample of a subject exposed to the pathogen.

6. The method of claim 1, wherein the assay mixture further comprises a third serotype of the pathogen and a fourth serotype of the pathogen.

7. The method of claim 6, wherein the assay mixture further comprises a third detectable label that specifically binds to the third serotype of the pathogen, and a fourth detectable label that specifically binds to the fourth serotype of the pathogen.

8. The method of claim 7, wherein the first, second, third and fourth detectable labels each comprise a distinctly detectable label.

9. The method of claim 8, wherein the evaluating comprises evaluating the assay mixture for the presence of each of the distinctly detectable labels.

10. The method of claim 8, wherein the distinctly detectable label is a fluorescent label.

11. The method of claim 1, wherein the pathogen comprises a virus or bacterium.

12. The method of claim 1, wherein the antibody is specifically bound to a capture probe.

13. The method of claim 12, wherein the capture probe is present on a surface of a substrate.

14. A method of determining serotype specificity of an anti-dengue virus antibody that binds to a serotype of a dengue virus, the method comprising:
specifically binding the anti-dengue virus antibody to a capture probe on a surface of a substrate to produce an anti-dengue virus antibody-capture probe complex;
contacting the anti-dengue virus antibody-capture probe complex with an assay mixture comprising:
a first, second, third and fourth serotype of the dengue virus;
a first detectable label that specifically binds to the first serotype of the dengue virus;
a second detectable label that specifically binds to the second serotype of the dengue virus;
a third detectable label that specifically binds to the third serotype of the dengue virus; and
a fourth detectable label that specifically binds to the fourth serotype of the dengue virus;
evaluating the assay mixture for the presence of an anti-dengue virus antibody-dengue virus-detectable label binding complex to determine the serotype specificity of the anti-dengue virus antibody.

15. A kit comprising:
at least first and second serotypes of a pathogen;
a first detectable label that specifically binds to the first serotype of the pathogen; and
a second detectable label that specifically binds to the second serotype of the pathogen.
